(19) United States
(12) Patent Application Publication
(10) Pub. No.: US 2007/0059777 A1
(43) Pub. Date: Mar. 15, 2007

(54) Thrombin-induced fibrinogen binding for the detection of risk of bleeding disorders

(75) Inventors: Fern Tablin, Davis, CA (US); Jeffrey W. Norris, Davis, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: REGENTS OF THE UNIVERSITY OF CALIFORNIA, Oakland, CA (US)

(21) Appl. No.: 11/510,271

(22) Filed: Aug. 24, 2006

Related U.S. Application Data
(60) Provisional application No. 60/711,713, filed on Aug. 25, 2005.

(51) Int. Cl. G01N 33/567; G01N 33/53

(52) U.S. Cl. 435/7.2

(57) ABSTRACT

This invention provides a methods of detecting a bleeding disorder in mammals where the bleeding disorder is characterized by normal fibrinogen binding to ADP-activated platelets, but decreased fibrinogen binding to thrombin-activates platelets.

### CROSS-REFERENCE TO RELATED APPLICATIONS

This application claims benefit of U.S. provisional application No. 60/711,713, filed Aug. 25, 2005, which application is incorporated by reference.

### STATEMENT AS TO RIGHTS TO INVENTIONS MADE UNDER FEDERALLY SPONSORED RESEARCH AND DEVELOPMENT

This invention was made with Government support under Grant No. N66001-00-C-8048, awarded by the Department of the Navy. The Government has certain rights in this invention.

### BACKGROUND OF THE INVENTION

Several cases of inherited coagulation defects have been reported in horses; however, there is only one report of an equine platelet dysfunction resulting in a bleeding diathesis (see, Sutherland, et al., Aust Vet J 66:366-370, 1989). In other species, e.g., humans, a number of heritable platelet disorders are known that are more prevalent in closely related populations (see, e.g., the review by Cattaneo, J Thromb Haemost 1:1628-36, 2003).

Recently, coagulation defects in a highly inbred thoroughbred filly with a severe bleeding diathesis have been described (Fry, et al., J. Vet. Internal Medicine 19:359-362, 2005). This subject had a template bleeding time consistently greater than two hours. Bleeding incidents could be successfully treated with the antifibrinolytic agent ε-amino-caproic acid, but were not resolved with whole blood transfusions. Results of standard clinicopathologic laboratory tests used to assess the coagulation system, including platelet concentration, prothrombin time, partial thromboplastin time, antithrombin III and measurement of serum coagulation factors (vWF, factors VIII, IX, XI, XII) were unremarkable. The function of the subject's platelet rich plasma (PRP) by aggregometry using a range of agonists was also evaluated (Fry, et al., supra). Although slightly diminished aggregation in response to thrombin was observed, the response of the subject's PRP to other agonists was unremarkable. The finding of aggregation in the subject's platelets in response to most agonists ruled out a Glannzmann-like thrombasthenia (Sutherland, et al., supra).

Defective aggregation and clotting due to platelet dysfunction often results from problems with the integral membrane proteins that mediate this process (Shattil & Newman, Blood 104:1606-1615, 2004). Fry et al. supra, reported that several tests of adhesion with the subject's platelets were normal, including their ability to retract a clot and adhere to fibrinogen substrates. The presence of key integral membrane proteins (αIIbβIIIa, GPIb, GPVI) on the subject's platelets was also normal in evaluations performed by Western Blot. In view of the largely normal clinical pathology tests, there was no method to readily diagnose this disorder in other horses, such as progeny of the affected animal.

The present invention is based on the discovery of altered properties of platelet function, e.g., altered fibrinogen binding properties, in the subject's platelets. Accordingly, the invention provides methods of diagnosing the presence of a bleeding defect in mammals for example, horses. The methods also provide a detection method for identifying animals at risk for a bleeding disorder, in both the general population and in particular populations, such as inbred populations, e.g., thoroughbred horses.

### BRIEF SUMMARY OF THE INVENTION

The present invention is based on the discovery of a previously unidentified bleeding disorder in mammals, e.g., horses. The bleeding disorder is characterized by a decrease in fibrinogen binding to platelets from the subject animal in response to a strong agonist.

Thus, the invention provides a method of detecting a risk for a bleeding disorder in a horse, the method comprising detecting a decrease in fibrinogen binding to platelets obtained from the horse where the platelets are activated by a strong agonist. The platelets employed in the assay are typically washed platelets. In some embodiments, the invention also comprises a step of determining fibrinogen binding to platelets isolated from the horse, where the platelets are activated by a weak agonist

The method can be performed on any horse, but in some embodiments, the horse is an offspring of a horse that has a bleeding disorder. The horse can be a racing horse, e.g., a thoroughbred, a quarter horse, or others; or can be a work horse or recreational horse.

Often when practicing the invention, the strong agonist is thrombin or thrombin receptor agonist peptide (TRAP). In embodiments in which fibrinogen binding to platelets in response to a weak agonist is also determined, the weak agonist is often ADP or ristocetin.

The level of fibrinogen binding can be detected using flow cytometry to detect labeled fibrinogen binding to the isolated platelets. The fibrinogen can be labeled with any detectable label. Typically, the fibrinogen is labeled with a fluorescent label. Detection of fibrinogen binding can also be performed using an immunological method employing an antibody to fibrinogen. In such an embodiment, the antibody to fibrinogen is typically labeled, e.g., with a fluorescent label or a label that has enzyme activity.

In another aspect, the invention provides a method of detecting a risk for a bleeding disorder in a mammal, the method comprising detecting a decrease in fibrinogen binding to platelets, typically washed platelets, obtained from the mammal that are activated by strong agonist; and detecting normal fibrinogen binding to platelets from the mammal that are activated by a weak agonist, thereby detecting an increased risk for a bleeding disorder in the mammal. In some embodiments, the mammal is an offspring of a mammal with a bleeding disorder. Preferably, the strong agonist is thrombin or TRAP. The weak agonist is often ristocetin or ADP. The level of fibrinogen binding is conveniently detected using flow cytometry to determine the level of binding of a labeled fibrinogen, e.g., a fluorescently labeled fibrinogen. In other embodiments, fibrinogen binding is detected using an antibody to fibrinogen.

In another aspect, the invention provides a method of detecting a risk for a bleeding disorder in a horse that has normal levels of glycoprotein IIb-IIIa, the method comprising detecting a decrease in aggregation of washed platelets in response to a strong agonist; and detecting normal aggregation of washed platelets from the horse that are activated by a weak agonist, thereby detecting an increased risk for a bleeding disorder in the mammal. The method often includes a step that assesses the levels of glycorpoteins IIb-IIIa. The strong agonist is often thrombin or TRAP. The weak agonist is often ristocetin or ADP. The horse can be any horse and in some embodiments, is the offspring of a horse that has a bleeding disorder. In other embodiments, the horse is a race horse, such as a thoroughbred race horse.

### BRIEF DESCRIPTION OF THE DRAWINGS

FIG. 1 provides representative data showing thrombin generation by equine platelets. Thrombin production was determined by the hydrolysis of S-2238. Time 0 is the initiation of thrombin production. Least-squares fits were used to determine the rates of thrombin production by resting control platelets (open circles, 1.6±0.4 U/min, R²=0.79), activated control platelets (open squares, 11.5±0.1 U/min, R²=0.9997), and activated platelets from the mare (closed circles, 5.5±0.7 U/min, R²=0.94). Values are expressed as the mean amount of thrombin produced by 1×10⁸ platelets at a concentration of 2.5×10⁷/ml.

FIG. 2 provides exemplary data showing scatter distributions of equine platelets activated with thrombin. Each panel in this figure is a forward scatter (Log FSC) vs. side scatter (Log SSC) plot for equine platelets as determined by flow cytometry. Panel A is a representative platelet distribution for control platelets; panel B shows platelets from the mare; and panel C shows control platelets to which both fibrinogen and the inhibitory peptide GPRP were added prior to activation. The color coding is a logarithmic scale corresponding to the number of platelets at each position in the diagram; black has the minimum number of platelets and the dark gray center the maximum.

FIG. 3 provides exemplary data showing fibrinogen binding to equine platelets activated with thrombin. Panel A is a typical histograms of Oregon Green-labeled fibrinogen bound to platelets from a control horse (solid line) and the mare (dotted line) 30 min after activation with 0.1 U/ml thrombin. Panel B indicates the relative amount of Oregon Green-labeled fibrinogen bound to platelets from the mare, colt, filly, and control horses. Average intensity of Oregon Green-labeled fibrinogen for platelets from each of the subjects was normalized to that of the controls; the normalized error bars represent SEM.

### DETAILED DESCRIPTION OF THE INVENTION

Introduction

The current invention is based on the discovery of a previously unknown bleeding disorder in mammals, e.g., horses. The bleeding disorder is characterized by loss of reactivity, e.g., aggregation, of washed platelets in response to strong agonists such as thrombin. Other agonists retain the ability to stimulate washed platelet reactivity. The bleeding disorder is often an inherited disorder and thus in some embodiments, detection is performed in particular populations of animals, e.g., thoroughbred race horses.

The reactivity of washed platelets is conveniently determined by assessing fibrinogen binding in response to a strong agonist. Fibrinogen binding is also typically determined in response to activation of platelets by a weak agonist. Thus, the invention also provides methods of measuring fibrinogen binding in response to one or more agonists to diagnose a bleeding disorder.

Assays for Platelet Reactivity

Platelets are prepared using standard methods. Platelet-rich plasma is obtained and the platelets washed. Platelet reactivity in response to strong (irreversible) and weak (reversible) agonists is then measured to determine whether the subject has a bleeding disorder in accordance with the invention.

Agonists

Platelet agonists employed in the present invention “activate” normal platelets for aggregation by converting the platelets from their normal resting state into a state suitable for subsequent aggregation. The bleeding disorder described herein is characterized by differential abilities of strong agonist vs. weak agonists to stimulate platelet reactivity, as reflected, e.g., by reduced fibrinogen binding to thrombin-stimulated platelets vs. fibrinogen binding to ADP-stimulated platelets.

In the context of this invention, a “strong” agonist refers to an agonist such as thrombin, which irreversibly activates platelets, i.e., stimulates the release of the contents of platelet granules at physiological concentrations. Upon activation, platelets release ADP and TXA2 (which activate additional platelets), serotonin, phospholipids, lipoproteins, and other proteins important for the coagulation cascade. Any strong agonist for use in this invention can be identified by its ability to irreversibly activate platelets. For example, secretion of platelet granule contents can be used as a measure of irreversible activation. This can be directly observed microscopically. Alternatively, secretion can be measured biochemically, for example, by measuring increases in the level of serotonin in the buffer.

Strong agonists include thrombin, and TRAP (thrombin receptor agonist peptide). These agonists can be used at a wide range of concentrations; in typical embodiments, thrombin is used in an amount of about 0.1 NIH-U/ml, but can be employed in lower amounts, e.g., about 0.01 NIH-U/ml. As appreciated by one of skill in the art, thrombin amount is often reported as unit/ml as an indicator of activity. In other instance, thrombin concentration can be represented in nM concentrations, but this does not provide the enzyme activity. Usually, agonist are employed at concentrations that approximate physiological concentrations.

Other platelet activating agents can also be used. In some instances, collagen or convulxin or collagen related peptide (CRP) in combination with thrombin or TRAP are strong agonists. For example, thrombin can be used at a concentration of 5 nM or more with collagen at a concentration of about 20 μg/ml. Convulxin can be used at a wide range of concentrations (0.5 ng/ml is the lowest to be tested; see Alberio et al. Blood. 5:1694-1702, 2000). A23197 and other ionophores for divalent cations as well as any of those molecules in combination with thrombin will also have similar effects. Accordingly, a strong agonist as used in this invention, refers to any agents, alone or in combination with another agent, that irreversibly activates platelets.

Weak Agonists

In the diagnostic methods of the invention, the reactivity of platelets is also frequently determined in response to weak agonists, such as ADP, low-dose collagen, U46619 (an endoperoxide analog), epinephrine, ristocetin, thromboxane A2, and the like. These agonists act reversibly on platelets, i.e., treatment does not lead to release of the contents of alpha granules present in the platelets. Accordingly, a weak agonist can be identified by measuring whether treatment of platelets with a physiological concentration of the weak agonist leads to release of the contents of the alpha granules.

Platelet Response

The reactivity of platelets isolated from subjects to be tested can be assessed using any number of assays. When assaying a parameter such as aggregation, washed platelets are typically employed. For example, platelet reactivity of platelets isolated from platelet poor plasma and then washed can be assessed by determining aggregation in response to various agonists, including both strong and weak agonist. Aggregation assays of this sort are well known in the art. For example, optical aggregometry can be used. In other embodiments, aggregation is assessed using flow cytometry.

In preferred embodiments, platelet samples from the subject mammals are assessed for the ability to bind fibrinogen. During platelet activation, glycoprotein IIb-IIIa undergoes a confirmation change such that it can bind fibrinogen. The new disease described herein is characterized by decreased fibrinogen binding in response to irreversible agonists such as thrombin, but unaltered fibrinogen binding in response to agonists such as ADP, which stimulate reversible platelet aggregation. In typical embodiments, fibrinogen binding is conveniently assessed using flow cytometric assays.

An exemplary flow cytometric assay is provided in the Examples section. Briefly, labeled fibrinogen, typically fluorescently labeled, is incubated with platelets that have been treated with an agonist. At various time points the amount of fibrinogen binding to platelets is determined. Control horse platelets rapidly bind fibrinogen in response to strong agonists, whereas fibrinogen binding is much slower in affected animals. Fibrinogen binding can be measured at one time point or over a time course, such as a time course of from five to sixty minutes. Typically, fibrinogen binding is measured at a time point that effectively resolves differences in level of fibrinogen binding to platelets from normal and affected animals, e.g., at about 30 minutes. For example, thrombin-induced fibrinogen binding to platelets from a normal animal reaches an endpoint after 15 minutes. In contrast, affected animals may have a delayed time course such that after forty five minutes, thrombin-induced platelets bind only as much fibrinogen as control platelets after five minutes.

Animals that are considered to have a diagnostic presence of the bleeding disorder when the level of strong agonist, e.g., thrombin,-induced fibrinogen binding is below about 80% of the level of strong agonist-induced fibrinogen binding in normal control animals. Typically, the levels of binding are less than 70% of control levels of binding and can be less than 30% or 20% of the binding in control animals at a time point that resolves the differences in bleeding. Levels of fibrinogen binding to platelets in response to a weak agonist such as ADP are comparable, e.g., 80% or greater, of the binding levels of fibrinogen to ADP-treated control platelets.

A control platelet sample from a normal animal is typically analyzed along with the sample from the subject animal. However, in some instances, a control value from a normal animal can be used as the normal control reference, thus fibrinogen binding to a control normal sample in the presence or absence of a strong and/or weak agonist need not always be assessed concurrently with the fibrinogen binding to the patient sample.

Fibrinogen binding is often detected using labeled fibrinogen. The fibrinogen can be directly labeled with any detectable label. In some embodiments, the label is a fluorescent label that can be conveniently detected. In other embodiments, fibrinogen binding is detected indirectly, e.g., using a labeled antibody that specifically binds to fibrinogen. Such detectable labels and detection systems are well known to those in the art. For example, an antibody can be labeled with a fluorescent label or a label that is detectable by enzymatic activity.

The fibrinogen need not be from the same species as the test subject, so long as the fibrinogen employed binds to activated normal platelets from the test animal species. For example, in some embodiments, labeled human fibrinogen can be used to detect horse platelet reactivity in response to agonists.

Detecting Levels of Glycoprotein IIb-IIIa

The fibrinogen receptor is a heterodimer consisting of the plasma-membrane glycoproteins (GP) IIb and IIIa. Although the GPIIb-IIIa complex is present on the surface of unstimulated platelets, it binds fibrinogen only after platelet activation. In some embodiments, the invention can optionally include a step of detecting the level of glycoprotein IIb-IIIa. In diagnosing the novel bleeding disorder in horse described herein, the level of platelet IIb-IIIa is that of normal platelets from a control. Typically, the subject animal exhibits at least 70%, more often at least 80%, or 90% or greater level of GPIIb-IIIa compared to normal controls.

GPIIb-IIIa is assayed using techniques well known in the art. Typically immunoassays using an antibody that interacts with one of the subunits is employed. Antibodies are commercially available (see, e.g., the sources listed in Fry et al., J. Vet. Intern. Med. 19:359-362, 2005). Platelets from normal control animals are generally evaluated concurrently with the test platelet sample. However, in some embodiments, the normal control reference value may be obtained from a normal sample that is not concurrently evaluated with the test platelet sample.

Subject Populations

Any mammal can be tested for the presence of a bleeding disorder or a propensity for a bleeding disorder as described herein. Mammals include horses, dogs, cats, cattle, sheep, goats, and the like. Often, the bleeding disorder is diagnosed in a particular subpopulation of the mammals that is extensively inbred.

In preferred embodiments, horses are evaluated for the bleeding disorder. Any horse can be tested, but often particular horse populations, e.g., thoroughbred horses, quarter horses, racing appaloosa, and the like are evaluated. Often, horses that have been observed to exhibit impairments in control of bleeding are tested. In other embodiments, horses that are related, e.g., offspring, of affected animals are evaluated for the presence of this defect in platelet activation.

The method of the present invention can also be used to assess the efficacy of a course of treatment. For example, a mammal, e.g., a horse, with a bleeding disorder as described herein, can be assessed for levels of strong agonist-induced fibrinogen binding to platelets in animals undergoing treatment. An increase in thrombin-induced fibrinogen binding to platelets from an animal undergoing treatment indicates efficacious treatment.

### EXAMPLES

### Example 1

### Characterization of a Bleeding Defect in a Thoroughbred Horse

Platelet reactivity was evaluated in a horse that has a bleeding defect. Washed platelets from the subject, offspring, and control horses were examined for the establishment of a phosphatidylserine (PS)-rich outer leaflet of the platelet membrane, the generation of thrombin, and the binding of fibrinogen in response to activation with thrombin.

An important prerequisite step in the common pathway involves the response of platelets to an increase in intracellular calcium by translocating PS from the inner to the outer leaflet of the platelet plasma membrane. Calcium mobilization was the same in platelets from controls and the subject mare (data not shown). This PS-rich outer membrane forms the foundation for the procoagulant surface and is required for normal assembly of prothrombinase (Dachary-Prigent, et al., Blood 81:2554-2565, 1993; Sims, et al., J Biol Chem 264:17049-17057, 1989). FITC-labeled annexin-V was used to detect PS translocation to the outer leaflets of platelets from control horses, the subject, and both offspring (Dachary-Prigent, et al., Blood 81:2554-2565, 1993; Kingston, et al., Am J Vet Res 63:513-519, 2002). Typically, 20% of resting platelets bind annexin-V, which increases to 70% after activation with 0.1 U/ml thrombin. The change does not depend on the dose of thrombin, between 0.1 and 1 U/ml, used to activate the platelets. The translocation of PS can be quantified by the increase in the mean fluorescent intensity of FITC-labeled annexin-V on activated platelets as compared to resting platelets. At 1 U/ml of thrombin, there was not a significant difference between the amounts of annexin-V bound by control platelets and either the subject's platelets (P=0.241) or those from her colt (P=0.448). Platelets from the subject's filly bound significantly more FITC-labeled annexin-V than control platelets (P=0.033), but this difference wasn't observed at lower concentrations of thrombin (P=0.056 at 0.5 U/ml) and is of uncertain significance. These data strongly suggest that the subject's platelets as well as those from her offspring have normal numbers of binding sites for prothrombinase.

A functional prothrombinase complex converts prothrombin to thrombin and relies on the proper assembly of factors Va and Xa on the PS-enriched outer membrane of the activated platelet. Factor Xa catalyzes the reaction, but is inefficient unless it binds factor Va, which binds PS. In humans, factor Va is found in both the plasma and the platelet α-granules (Gunson, et al., J Amer Vet Assoc 193:102-106, 1988). Platelet factor Va is structurally different from the plasma factor Va and has been suggested to be the more important of the two for normal hemostasis.

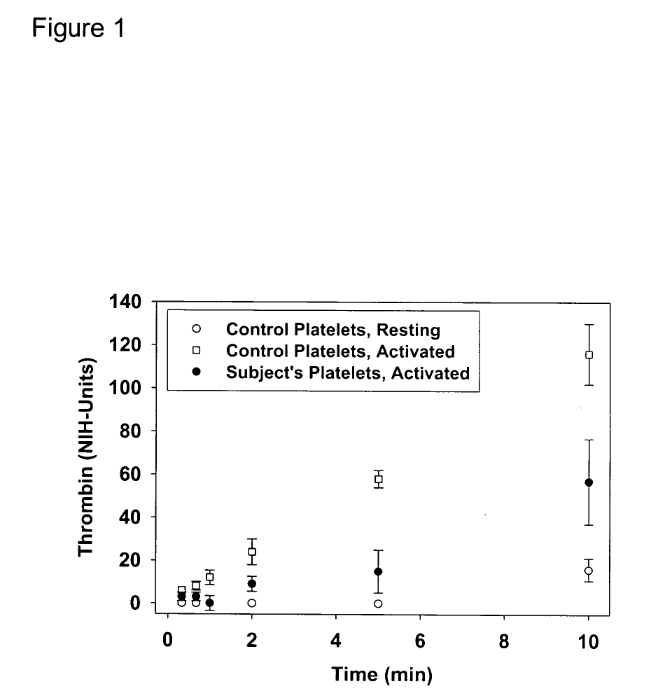

The amount of thrombin produced under conditions where only endogenous factor Va was available to form the prothrombinase complex was measured. Control platelets immediately began converting prothrombin to thrombin at a constant rate that averaged 1.93±0.23 U/min (FIG. 1). In contrast, the subject's platelets did not produce measurable amounts of thrombin until 10 min after initiation of the reaction. On average, the subject's platelets produced thrombin at half the rate of control platelets (0.95±0.33 U/min) and generated significantly less total thrombin than control platelets.

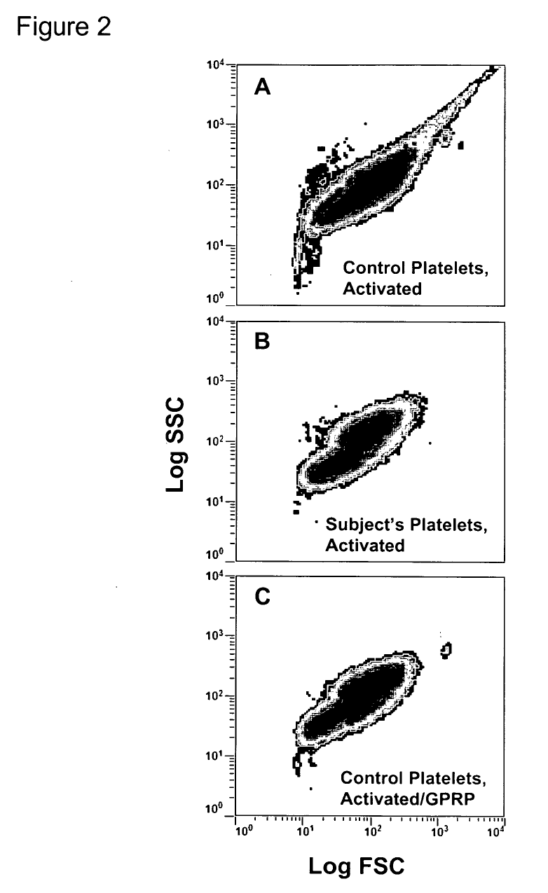

Thrombin directly affects platelet aggregation by proteolyzing fibrinogen, which then polymerizes to form the fibrin meshwork that provides the scaffold for a clot. Flow cytometry was used to observe aggregates formed by washed platelets that had been incubated with purified human fibrinogen. Platelet aggregation was determined by the light scatter properties of the samples. Control horse platelets, stimulated with 0.1 U/ml thrombin, formed an elongated distribution extending one log unit beyond resting platelets in both forward and side scatter dimensions, which occurs due to aggregation of the platelets (FIG. 2A). The subject's platelets, treated in an identical manner, failed to produce an aggregate distribution (FIG. 2B). This failure is consistent with the reduction in prothrombinase activity reported here and the reduced aggregation of the subject's PRP in response to thrombin that was previously reported (Fry, et al., J. Vet. Internal Medicine 19:359-362, 2005).

Aggregation of normal platelets can be inhibited by blocking the polymerization of fibrinogen with the peptide GPRP. This peptide binds directly to the site at which fibrinogen molecules self-associate (Laudano & Doolittle, Proc Natl Acad Sci USA 75:3085-3089, 1978). The elongated distribution seen by flow cytometry for activated control platelets was not observed when they were activated in the presence of GPRP. The GPRP treated control platelets had a distribution similar to platelets from the subject that were activated without GPRP present (FIG. 2C). This result further supports our hypothesis that a defect in the common pathway is the underlying cause of the subject's bleeding diathesis.

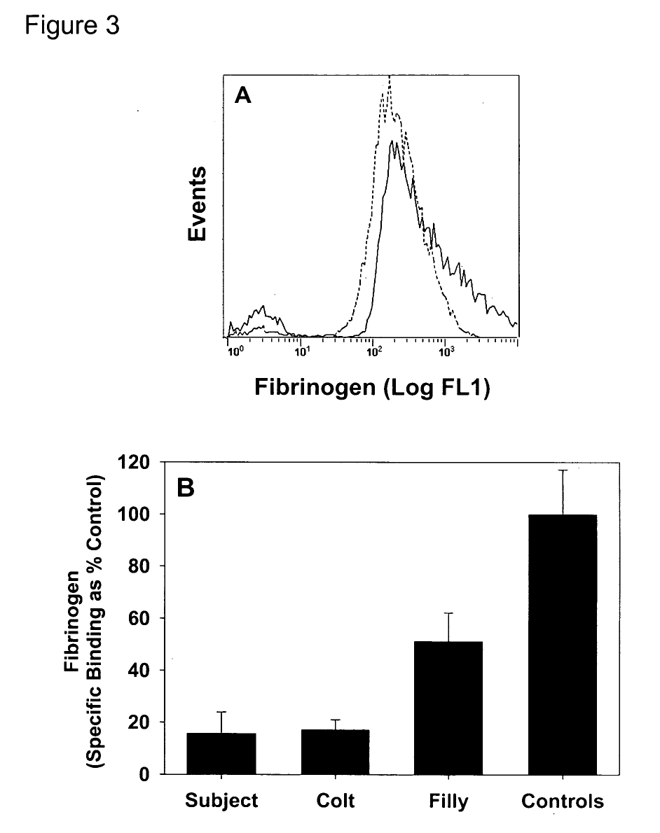

Aggregation involves the polymerization of multiple fibrinogen molecules, and Oregon Green-labeled fibrinogen can be used as a simple and sensitive tool for quantifying the amount of aggregation. The amount of labeled fibrinogen bound to a platelet or platelet aggregate is proportional to its fluorescence intensity (see, e.g., Faraday, et al., J Lab Clin Med 123:728-740, 1994). Flow cytometry histograms of platelets activated in the presence of Oregon Green-labeled fibrinogen demonstrate that the intensity of subject's platelets was approximately 20% that of control platelets (FIG. 3A). This binding was quantified for platelets from the subject and her offspring as a percent of fibrinogen binding to control platelets (FIG. 3B). Like the subject, her colt bound approximately 20% as much fibrinogen as control platelets and failed to produce an elongated distribution that could be detected by light scatter properties (data not shown). Platelets from the subject's filly bound half as much fibrinogen as control platelets and produced an elongated distribution. These results suggest that the subject's disorder is heritable and transmitted in an autosomal fashion.

As a control for the assay the binding of Oregon Green-labeled fibrinogen to platelets stimulated with ADP was evaluated. ADP, unlike thrombin, directly activates the (αIIbβIIIa integrin, the platelet fibrinogen receptor, without inducing significant aggregation at low concentrations of platelets (<1×10⁸ platelets/ml) (Shattil, et al., J Biol Chem 260:11107-11114, 1985). The binding of Oregon Green-labeled fibrinogen to ADP-stimulated platelets from both controls and the subject was the virtually identical when analyzed by flow cytometry (data not shown). These results are consistent with previous report that the subject's platelets express normal quantities of αIIbβIIIa (Fry, et al., J. Vet. Internal Medicine 19:359-362, 2005).

Materials and Methods

Animals—The subject horse, two offspring (produced via embryo transfer) and 3 clinically normal horses housed at the Center for Equine Health, University of California—Davis were used in these studies under institutionally approved protocols.

Platelet preparation—Blood samples were collected into ACD-A vacutainer tubes and transported to the laboratory for processing at room temperature. Within 60 min of collection, blood was centrifuged in 17×120 mm conical, polypropylene tubes (200×g, 15 min, room temperature (25° C.)). The platelet-rich plasma (PRP) was transferred to a 17×100 mm round-bottom, polypropylene tube, 10 μg/ml PGE₁ (final) was added, and the tube was centrifuged (400×g, 15 min, room temperature). The platelet pellet was suspended in 10 ml Tyrode's-HEPES (12 mM NaHCO₃, 138 mM NaCl, 2.9 mM KCl, 10 mM HEPES, 10 μg/ml PGE₁, pH 7.2), pelleted again by a second centrifugation under the same conditions, and suspended in Tyrode's-HEPES at 1×10⁸ cells/ml unless otherwise noted. When indicated, CaCl₂ was added to platelets in five equal aliquots at five minute intervals from a 10 mM stock in Tyrode's-HEPES. All cell counts were determined using an automated blood counter.

Flow cytometry—All data were collected with an FC500 flow cytometer. Forward and side scatter voltages were set to detect machine noise, which was removed during subsequent analyses. Binding experiments with FITC-labeled annexin-V and Oregon Green-labeled fibrinogen in response to ADP, the potential of the FL1 detector was set such that 95% of control platelet autofluorescence was binned in the first log of the detector response. For experiments with Oregon Green-labeled fibrinogen binding in response to thrombin, the potential of the FL1 detector was arbitrarily set to 500V to prevent saturation of the detector.

Detection of surface phosphatidylserine—Washed platelets (5×10⁷ cells/ml) in Tyrode's-HEPES plus 2 mM CaCl₂ were activated with thrombin (0.1, 0.5, and 1 U/ml) for 15 min at 37° C. 5×10⁵ platelets were removed from the reaction, diluted to 100 ml with Tyrode's-HEPES plus 2 mM CaCl₂, and labeled with FITC-labeled annexin-V according to the manufacturer's recommendation. After incubating the sample for an additional 10 min at 37° C., the sample was analyzed by flow cytometry.

Prothrombinase activity assay—Prothrombinase activity was detected on washed platelets at 5×10⁷ cells/ml in Tyrode's-HEPES buffer plus 2 mM CaCl₂ and 1 mM MgCl₂. Platelets were activated with 0.1 U/ml of thrombin and incubated for 15 min at 37° C. prior to the addition of 1 nM bovine factor Xa. The reaction was incubated for an additional 10 min at 37° C. to allow for maximal factor Va release and factor Xa binding (Baruch, et al., Eur J Biochem 154:213-218, 1986; Tracy, et al., J Biol Chem 256:743-751, 1981). Thrombin generation was initiated with the addition of 5 μM (final) bovine prothrombin. Aliquots were then removed at 20 sec, 40 sec, 1 min, 2 min, 5 min and 10 min and diluted with nine volumes of ice-cold stop solution (Tyrode's-HEPES buffer containing 10 mM EDTA). These “stopped” aliquots were then added to 0.1 mM (final) S-2238, a chromogenic substrate for thrombin that increases in absorbance at a rate proportional to the amount of thrombin present in the aliquot. For each time point, the absorbance was measured continuously by spectrophotometry, and the rate was calculated by averaging the change over a 90 sec interval during the linear phase of the reaction, between the initial mixing phase and before the substrate became depleted. A standard curve, using defined amounts of thrombin, was used to convert these rates to amounts of thrombin generated by the platelets.

Assessment of fibrinogen binding and platelet aggregation—Washed platelets (5×10⁷ cells/ml) in Tyrode's-HEPES buffer plus 2 mM CaCl₂ were incubated with Oregon Green-labeled fibrinogen (3 μg/ml) for one minute at room temperature (25° C.) prior to activation. Platelets were activated with either 50 μM ADP or 0.1 U/ml thrombin and evaluated by flow cytometry after a 30 min incubation at 37° C. In some experiments on control horse platelets, fibrinogen polymerization on thrombin-activated platelets was inhibited with 1 mM GPRP, a peptide that sterically inhibits fibrinogen polymerization (Laudano & Doolittle, Proc Natl Acad Sci USA 75:3085-3089, 1978). The inhibitor was added prior to activating the platelets with thrombin

Statistical analysis—In all experiments, means and standard errors of the means are calculated from values determined from three normal horses, each assayed once. For the subject mare and her offspring, the means and standard errors of the means are calculated from the values determined for three replicates on different days. For each experiment, the statistical significance of differences between the control and subject animals was determined by two-tailed t-test. In all figures, the error bars represent the standard error of the mean.

### Example 2

### Screening of a Horse Population for the Presence of the Bleeding Defect

A horse population of 446 animals was evaluated for the presence of the bleeding defect described in Example 1. Blood samples were collected into ACD-A vacutainer tubes and transported to the laboratory within 48 hrs at ambient temperature, which varied between 1 and 52° C. Platelets were prepared and washed as described in the Materials and Methods section for Example 1.

Fibrinogen binding was assessed in response to thrombin. Washed platelets (5×10⁷ cells/ml) in Tyrode's-HEPES buffer plus 2 mM CaCl₂ were incubated with labeled fibrinogen (3 μg/ml) for one minute at room temperature (25° C.) prior to activation. Platelets were activated with 0.1 U/ml thrombin and evaluated by flow cytometry after a 30 min incubation at 37° C. All data were collected with an FC500 flow cytometer. Forward and side scatter voltages were set to detect machine noise, which was removed during subsequent analyses.

The results showed that 2 horses in 446 consistently had decreased fibrinogen levels identical to that of the index case in all trials of those animals (n=2 and n=4). Thus, the bleeding defect can be detected in horse populations.

### SUMMARY

The results exemplified herein demonstrate a defect in a subject horse platelets that can be used to diagnose this bleeding disorder. Fibrinogen binding in response to thrombin activation of platelets provides a sensitive method for detecting this platelet dysfunction. Using this assay we observed that the male offspring had reduced fibrinogen binding, suggesting that the platelet dysfunction can be genetically transmitted in an autosomal manner.

The results indicate that the defect in the subject's platelets is due to an alteration in the enzymatic reactions of the common pathway on the platelet surface. Primary evidence of the altered enzymatic reaction is the reduced prothrombinase activity observed in the subject animal, which correlates with the decreased aggregation of the subject's platelets in response to thrombin (Fry et al, supra).

Defects in the “common pathway” of coagulation have been identified for a number of elements in the process, including the creation of a procoagulant surface, assembly of enzymatic complexes, production of thrombin and subsequent fibrin polymerization. A necessary first step in the creation of a procoagulant surface is the translocation of PS from the inner leaflet to the outer leaflet of the platelet membrane. Scott syndrome is an autosomal recessive defect in PS translocation (Dachary-Prigent, et al. Blood 81:2554-2565, 1993) which has been characterized in both humans and German Shepard dogs J (Brooks, et al., Blood 99:2434-2441, 2002; Weiss et al., Am J Med 67:206-213, 1979). The defect is detected in patients with moderate to severe bleeding, normal platelet counts and normal platelet ultrastructure. It can be identified by reduced binding of FITC-labeled annexin-V (Dachary-Prigent, et al., Blood 81:2554-2565, 1993). This value was the same for control horses, the subject mare, and her offspring in the studies described herein above.

Factors V and X, which assemble into the prothrombinase complex, are other possible sources of the subject's genetic defect. Factor X is found in the plasma, while factor V can be derived from either the plasma or the platelet α-granules (Tracy, et al., Blood 60:59-63, 1982). Several genetic defects of factor X and plasma factor V have been characterized in humans and in all cases have a prolongation of either or both prothrombin time and partial thromboplastin time (Roberts & Zeitler, In: Colman, et al., eds. Hemostasis and Thrombosis 1st ed. Philadelphia: J B Lippincott Co, 1982;127-144). Plasma factor function of the subject was evaluated by standard coagulation tests (prothrombin time and partial thromboplastin time) and found to be unremarkable. In some cases, patients with plasma factor V defects also have a 70% or greater deficiency in factor VIII. The subject of this study has a clinically insignificant reduction (20% or normal) reduction in factor VIII.

The significantly decreased prothrombinase demonstrated in the subject's platelets could be attributed to a defect in platelet factor V. There are two defects in platelet factor V function that have been characterized in humans. Quebec platelet disorder results from the degradation of factor V by urokinase plasminogen activator (uPA), which is overexpressed in the α-granules of patients with this disorder (Kahr, et al., Blood 98:257-265, 2001). The increased levels of uPA are clinically detectable by increased levels of fibrinogen degradation products in plasma (Hayward, et al., Br J Haematol 97:497-503, 1997), which were not observed in plasma from the subject mare or her offspring (data not shown). Factor V-New York is a disorder characterized by a 50% reduction in prothrombinase activity, but no discernable degradation of α-granule proteins (Weiss, et al., Am J Hematol 2001;66:130-139, 2001). Precise characterization of the molecular defect in this subject, associated with decreased prothrombinase activity, requires further investigation.

Reduced prothrombinase activity could account for the slight reduction in aggregation of the subjects' platelets that was previously noted in response to 0.5 U/ml thrombin (Fry et al., supra). Control horse platelets treated with an identical amount of thrombin demonstrated irreversible aggregation, consistent with the studies of Weiss et al. (Vet Clin. Pathol. 19:35-39, 1990). However, these studies indicated that thrombin stimulated aggregometry yielded inconsistent results when comparing normal horses to horses with epistaxis due to EIPH.

In the experiments described above, formation of aggregates was determined by flow cytometry using a washed platelet preparation with the addition of human fibrinogen. In this system, aggregation defects of the subject's washed platelets were clearly observed and mimicked in control platelets by including GPRP in the reaction.

Binding of fibrinogen, e.g., Oregon Green-labeled fibrinogen, to washed platelets in response to low doses of thrombin (0.1 U/ml) is a sensitive and reproducible assay for detecting defects in platelet aggregation. Platelets stored overnight at room temperature as whole blood in ACD-A can be assayed without compromising detection capability. We have used this assay to identify fibrinogen binding defects in platelets from the subject's offspring.

This assay is useful for screening horses for congenital defects in the common pathway. Further study may show that the assay can be used to detect subclinical, acquired thrombopathies associated with drug administration, endotoxemia, colic or other inflammatory disease states.

All publications, patents, and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of detecting a risk for a bleeding disorder in a horse, the method comprising detecting a decrease in fibrinogen binding to platelets obtained from the horse, wherein the platelets are activated by strong agonist; thereby detecting an increased risk for a bleeding disorder in the horse.

2. The method of claim 1, further comprising detecting normal fibrinogen binding to platelets from the horse that are activated by a weak agonist.

3. The method of claim 1, where the method further comprises detecting normal levels of glycoprotein IIb-IIIa.

4. The method of claim 1, wherein the assay is performed on platelets from a horse that is the offspring of a horse with a bleeding disorder.

5. The method of claim 1, wherein the horse is a thoroughbred.

6. The method of claim 1, wherein the strong agonist is thrombin.

7. The method of claim 2, wherein the weak agonist is ristocetin or ADP.

8. The method of claim 7, wherein the weak agonist is ADP.

9. The method of claim 1, wherein the level of fibrinogen binding is detected by flow cytometry.

10. The method of claim 1, wherein the fibrinogen is labeled with a fluorescent label.

11. The method of claim 1, wherein the method comprises detecting the level of fibrinogen binding using an antibody to fibrinogen.

12. A method of detecting a risk for a bleeding disorder in a mammal, the method comprising detecting a decrease in fibrinogen binding to platelets obtained from the mammal that are activated by strong agonist, thereby detecting an increased risk for a bleeding disorder in the mammal.

13. The method of claim 12, further comprising a step of detecting normal fibrinogen binding to platelets from the mammal that are activated by a weak agonist.

14. The method of claim 12, wherein the assay is performed on platelets from a mammal that is the offspring of a mammal with a bleeding disorder.

15. The method of claim 12, wherein the strong agonist is thrombin.

16. The method of claim 12, wherein the weak agonist is ristocetin or ADP.

17. The method of claim 12, wherein the weak agonist is ADP.

18. The method of claim 12, wherein the level of fibrinogen binding is detected by flow cytometry

19. The method of claim 12, wherein the fibrinogen is labeled with a fluorescent label.

20. The method of claim 12, wherein the method comprises detecting the level of fibrinogen binding using an antibody to fibrinogen.

21. A method of detecting a risk for a bleeding disorder in a horse that has normal levels of glycoprotein IIb-IIIa, the method comprising detecting a decrease in aggregation of washed platelets in response to a strong agonist; and detecting normal aggregation of washed platelets from the horse that are activated by a weak agonist thereby detecting an increased risk for a bleeding disorder in the horse.
